(19) United States
(12) Patent Application Publication
(10) Pub. No.: US 2003/0158120 A1
(43) Pub. Date: Aug. 21, 2003

(54) Combination product comprising melagatran and a factor xa inhibitor

(75) Inventor: Christer Mattsson, Kungsbacka (SE)

Correspondence Address: ROPES & GRAY LLP, ONE INTERNATIONAL PLACE, BOSTON, MA, 02110-2624, US

(73) Assignee: AstrZeneca AB, Sodertalje (SE)

(21) Appl. No.: 10/297,537

(22) PCT Filed: Jun. 6, 2001
(86) PCT No.: PCT/SE01/01288

(30) Foreign Application Priority Data
Jun. 10, 2000 (GB) 0014136.6

(51) Int. Cl. A61K 31/397

(52) U.S. Cl. 514/19; 514/210.17

(57) ABSTRACT

There is provided a combination product comprising: (A) melagatran or a pharmaceutically-acceptable derivative thereof; and (B) a Factor Xa inhibitor or a pharmaceutically-acceptable derivative thereof, wherein each components (A) and (B) is formulated in admixture with a pharmaceutically-acceptable adjuvant, diluent or carrier, as well as the use of such a combination product in the treatment of a condition where anticoagulant therapy is indicated.

### FIELD OF THE INVENTION

This invention relates to a new combination of pharmaceutically-active compounds.

### BACKGROUND AND PRIOR ART

Blood coagulation is the key process involved in both haemostasis (i.e. the prevention of blood loss from a damaged vessel) and thrombosis (i.e. the formation of a blood clot in a blood vessel, sometimes leading to vessel obstruction).

Coagulation is the result of a complex series of enzymatic reactions.

One of the ultimate steps in this series of reactions is the conversion of the proenzyme prothrombin to the active enzyme thrombin.

Thrombin is known to play a central role in coagulation. It activates platelets, leading to platelet aggregation, converts fibrinogen into fibrin monomers, which polymerise spontaneously into fibrin polymers, and activates factor XIII, which in turn crosslinks the polymers to form insoluble fibrin. Furthermore, thrombin activates factor V and factor VIII leading to a “positive feedback” generation of thrombin from prothrombin.

International patent application WO 94/29336 discloses a group of thrombin-inhibiting compounds, including HOOC—CH₂—(R)Cgl-Aze-Pab-H (in which Cgl represents cyclohexylglycine, Aze represents S-azetidine-2-carboxylic acid and Pab-H represents 4-aminomethylamidinobenzene), which is also known as melagatran (see Example 1 of WO 94/29336). International Patent Application WO 97/23499 discloses prodrugs of inter alia melagatran.

Factor Xa is one of a cascade of proteases involved in the process of blood coagulation. Factor Xa is the preceding protease, which cleaves prothrombin to generate thrombin.

Certain compounds are known to possess Factor Xa inhibitory properties and the field has been reviewed by R. B. Wallis, Current Opinion in Therapeutic Patents, 1993, 1173-1179. It is known that two proteins, one known as antistatin and the other known as tick anticoagulant protein (TAP), are specific Factor Xa inhibitors which possess antithrombotic properties in various animal models of thrombotic disease.

It is also known that certain non-peptidic compounds possess Factor Xa inhibitory properties.

None of the above-mentioned documents disclose or suggest the administration of melagatran or a pharmaceutically-acceptable derivative thereof in conjunction with a Factor Xa inhibitor. We have now found, surprisingly, that administration of just such a combination gives rise to a notable synergistic anticoagulant effect.

### DISCLOSURE OF THE INVENTION

According to a first aspect of the invention there is provided a combination product comprising:

(A) melagatran or a pharmaceutically-acceptable derivative thereof; and

(B) a Factor Xa inhibitor or a pharmaceutically-acceptable derivative thereof,

wherein each of components (A) and (B) is formulated in admixture with a pharmaceutically-acceptable adjuvant, diluent or carrier.

The combination product according to the invention provides for the administration of melagatran (or derivative thereof) in conjunction with a Factor Xa inhibitor (or derivative thereof), and may thus be presented either as separate formulations, wherein at least one of those formulations comprises melagatran and at least one comprises Factor Xa inhibitor, or may be presented (i.e. formulated) as a combined preparation (i.e. presented as a single formulation including melagatran and Factor Xa inhibitor).

Thus, there is further provided:

(1) a pharmaceutical formulation including melagatran or a pharmaceutically-acceptable derivative thereof, and a Factor Xa inhibitor or a pharmaceutically-acceptable derivative thereof, in admixture with a pharmaceutically-acceptable adjuvant, diluent or carrier (which formulation is hereinafter referred to as a “combined preparation”); and

(2) a kit of parts comprising components:

(a) a pharmaceutical formulation including melagatran or a pharmaceutically-acceptable derivative thereof, in admixture with a pharmaceutically-acceptable adjuvant, diluent or carrier; and

(b) a pharmaceutical formulation including a Factor Xa inhibitor or a pharmaceutically-acceptable derivative thereof, in admixture with a pharmaceutically-acceptable adjuvant, diluent or carrier,

which components (a) and (b) are each provided in a form that is suitable for administration in conjunction with the other.

According to a further aspect of the invention, there is provided a method of making a kit of parts as defined above, which method comprises bringing a component (a), as defined above, into association with a component (b), as defined above, thus rendering the two components suitable for administration in conjunction with each other.

By bringing the two components “into association with” each other, we include that components (a) and (b) of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Thus, there is further provided a kit of parts comprising:

(I) one of components (a) and (b) as defined herein; together with

(II) instructions to use that component in conjunction with the other of the two components.

The kits of parts described herein may comprise more than one formulation including an appropriate quantity/dose of melagatran or derivative thereof, and/or more than one formulation including an appropriate quantity/dose of Factor Xa inhibitor or derivative thereof, in order to provide for repeat dosing. If more than one formulation (comprising either active compound) is present, such formulations may be the same, or may be different in terms of the dose of melagatran (or derivative) or Factor Xa inhibitor (or derivative), chemical composition and/or physical form.

A further aspect of the invention provides a method of treatment of a condition where anticoagulant therapy is indicated, which comprises administration of a pharmaceutical formulation including melagatran (or a pharmaceutically-acceptable derivative thereof), and a Factor Xa inhibitor (or a pharmaceutically-acceptable derivative thereof), in admixture with a pharmaceutically-acceptable adjuvant, diluent or carrier.

A further aspect of the invention provides a method of treatment of a condition where anticoagulant therapy is indicated (by which we mean where anticoagulation is required), which comprises administration of:

(a) a pharmaceutical formulation including melagatran or a pharmaceutically-acceptable derivative thereof, in admixture with a pharmaceutically-acceptable adjuvant, diluent or carrier; in conjunction with

(b) a pharmaceutical formulation including a Factor Xa inhibitor or a pharmaceutically-acceptable derivative thereof, in admixture with a pharmaceutically-acceptable adjuvant, diluent or carrier,

to a patient suffering from, or susceptible to, such a condition.

For the avoidance of doubt, as used herein, the term “treatment” includes therapeutic and/or prophylactic treatment.

With respect to the kits of parts as described herein, by “administration in conjunction with”, we include that respective formulations comprising melagatran (or derivative thereof) and Factor Xa inhibitor (or derivative thereof) are administered, sequentially, separately and/or simultaneously, over the course of treatment of the relevant condition, which condition may be acute or chronic.

Thus, in respect of the combination product according to the invention, the term “administration in conjunction with” includes that the two components of the combination product (melagatran/derivative and Factor Xa inhibitor/derivative) are administered (optionally repeatedly), either (in the case of a combined preparation) together, or (in the case of a kit of parts) sufficiently closely in time, to enable a beneficial effect for the patient, that is greater, over the course of the treatment of the relevant condition, than if either a formulation comprising melagatran/derivative, or a formulation comprising Factor Xa inhibitor/derivative, are administered (optionally repeatedly) alone, in the absence of the other component, over the same course of treatment. Determination of whether a combination provides a greater beneficial effect in respect of, and over the course of treatment of, a particular condition, will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration with the other component. When used in this context, the terms “administered simultaneously” and “administered at the same time as” include that individual doses of melagatran (or derivative thereof) and Factor Xa inhibitor (or derivative thereof) are administered within 48 hours (e.g. 24 hours) of each other.

“Pharmaceutically-acceptable derivatives” of melagatran and Factor Xa inhibitors includes salts (e.g. pharmaceutically-acceptable non-toxic organic or inorganic acid addition salts) and solvates. It will be appreciated that the term further includes derivatives that have the same biological function and/or activity as melagatran, or the Factor Xa inhibitor, as appropriate. Moreover, for the purposes of this invention, the term also includes prodrugs of melagatran or Factor Xa inhibitors. “Prodrugs” of melagatran, or Factor Xa inhibitors, include any composition of matter that, following oral or parenteral administration, is metabolised in vivo to form either melagatran, or the respective Factor Xa inhibitor, as appropriate, in an experimentally-detectable amount, and within a predetermined time (e.g. within a dosing interval of between 6 and 24 hours (i.e. once to four times daily)). For the avoidance of doubt, the term “parenteral” adminstration includes all forms of adminstration other than oral administration.

Prodrugs of melagatran that may be mentioned include those disclosed in international patent application WO 97/23499. Preferred prodrugs are those of the formula R¹O₂C—CH₂—(R)Cgl-Aze-Pab-OH (see the list of abbreviations above or in WO 97/23499), wherein R¹ represents C₁₋₁₀ alkyl or benzyl, such as linear or branched C₁₋₆ alkyl (e.g. C₁₋₄ alkyl, especially methyl, n-propyl, i-propyl, t-butyl and, particularly, ethyl) and the OH group replaces one of the amidino hydrogens in Pab.

Factor Xa inhibitors that may be used in the combination products according to the invention include those described in Current Opinion in Therapeutic Patents, 1993, 1173-1179 and in international patent applications WO 00/20416, WO 00/12479, WO 00/09480, WO 00/08005, WO 99/64392, WO 99/62904, WO 99/57096, WO 99/52895, WO 99/50263, WO 99/50257, WO 99/50255, WO 99/50254, WO 99/48870, WO 99/47503, WO 99/42462, WO 99/42439, WO 99/40075, WO 99/37304, WO 99/36428, WO 99/33805, WO 99/33800, WO 99/32477, WO 99/32454, WO 99/31092, WO 99/26941, WO 99/26933, WO 99/26932, WO 99/26919, WO 99/26918, WO 99/25720, WO 99/16751, WO 99/16747, WO 99/12935, WO 99/12903, WO 99/11658, WO 99/11617, WO 99/10316, WO 99/07732, WO 99/07731, WO 99/05124, WO 99/00356, WO 99/00128, WO 99/00127, WO 99/00126, WO is 99/00121, WO 98/57951, WO 98/57937, WO 98/57934, WO 98/54164, WO 98/46591, WO 98/31661, WO 98/28282, WO 98/28269, WO 98/25611, WO 98/24784, WO 98/22483, WO 98/16547, WO 98/16525, WO 98/16524, WO 98/16523, WO 98/15547, WO 98/11094, WO 98/07725, WO 98/06694, WO 98/01428, WO 97/48706, WO 97/46576, WO 97/46523, WO 97/38984, WO 97/30971, WO 97/30073, WO 97/29067, WO 97/24118, WO 97/23212, WO 97/21437, WO 97/08165, WO 97/05161, WO 96/40744, WO 96/40743, WO 96/40679, WO 96140100, WO 96/38421, WO 96/28427, WO 96/19493, WO 96/16940, WO 95/28420, WO 94/13693, WO 00/24718, WO 99/55355, WO 99/51571, WO 99/40072, WO 99/26926, WO 98/51684, WO 97/48706, WO 97/24135, WO 97/11693, WO 00/01704, WO 00/71493, WO 00/71507, WO 00/71508, WO 00/71509, WO 00/71511, WO 00/71512, WO 00/71515, WO 00/71516, WO 00/13707, WO 00/31068, WO 00/32590, WO 00/33844, WO 00/35859, WO 00/35886, WO 00/38683, WO 00/39087, WO 00/39092, WO 00/39102, WO 00/39108, WO 00/39111, WO 00/39117, WO 00/39118, WO 00/39131, WO 00/40548, WO 00/40571, WO 00/40583, WO 00/40601, WO 00/47207, WO 00/47553, WO 00/47554, WO 00/47563, WO 00/47578, WO 00/51989, WO 00/53264, WO 00/59876, WO 00/59902, WO 00/71510, WO 00/76970, WO 00/76971, WO 00/78747, WO 01/02356, WO 01/02397, WO 01/05784, WO 01/09093, WO 01/12600, WO 01/19788, WO 01/19795, WO 01/19798, WO 93/15756, WO 94/17817, WO 95/29189, WO 96/18644, WO 96/20689, WO 96/39380, WO 97/22712, WO 97/36580, WO 97/36865, WO 97/48687, WO 98/09987, WO 98/46626, WO 98/46627, WO 98/46628, WO 98/54132, WO 99/07730, WO 99/33458, WO 99/37643 and WO 99/64446; in U.S. Pat. Nos. 6,034,093, 6,020,357, 5,994,375, 5,886,191, 5,849,519, 5,783,421, 5,731,315, 5,721,214, 5,693,641, 5,633,381, 5,612,378, 6,034,127, 5,670,479, 5,658,939, 5,658,930, 5,656,645, 5,656,600, 5,639,739, 5,741,819, 6,057,342, 6,060,491, 6,080,767, 6,087,487, 6,140,351 and 5,646,165; in Japanese patent applications Nos. JP 99152269, JP 10017549, JP 10001467, JP 98017549, JP 00178243, JP 11140040, JP 12143623, JP 12204081, JP 12302765, JP 6327488 and JP 98001467; in European patent applications EP 937 723, EP 937 711, EP 874 629, EP 842 941, EP 728 758, EP 540 051, EP 419 099, EP 686 642, EP 1 016 663 and EP 529 715; and in German patent applications Nos. DE 19845153, DE 19835950, DE 19743435, DE 19829964, DE 19834751, DE 19839499, DE19900355, DE19900471 and DE 19530996, the specific and generic disclosures in all of which documents are hereby incorporated by reference.

Factor Xa inhibitors that may be mentioned also include those disclosed in international patent applications WO 96/10022, WO 97/28129, WO 97/29104, WO 98/21188, WO 99/06371, WO 99/57099, WO 99/57112, WO 00/47573, WO 00/78749, WO 99/09027 and WO 99/57113, the specific and generic disclosures in all of which documents are hereby incorporated by reference, as well as 4-{4-[4-(5-chloroindol-2-ylsulfonyl) piperazine-1-carbonyl]phenyl}pyridine-1-oxide and pharmaceutically-acceptable derivatives thereof, which may be prepared according to the method described in Example 1 below.

Preferred Factor Xa inhibitors include antistatin, tick anticoagulant protein and those known as SQ-311 and SQ-315 (see international patent application WO 98/57951); SN-292 (see international patent application WO 98/28282); SN429 and SN 116 (see international patent application WO 98/28269); RPR-208707 (see international patent application WO 98/25611 at Example 48); XU-817 (see international patent application WO 98/01428); SF-324 and SF-303 (see international patent application WO 97/23212); YM 60828 (see international patent application WO 96/16940 at Example 75); FACTOREX (see U.S. Pat. No. 5,783,421); SF-324 (see European patent application EP 874 629); DX9065A (see European patent application EP 540 051 at Example 39); 1-(4-amidinobenzyl)-4-(6-chloronaphthalene-2-ylsulfonyl)piperazin-2-one (see JP 12204081 at Example 2); M55555 (see international patent application WO 99/33805 at Example 39); DPC423 (1-(3-amidinophenyl)-2-(2′-aminosulfonyl[1,1′-biphenyl]4-ylaminocarbonyl)-4-bromopyrrole, see international patent application WO 98/28269); 3-(3,5-difluoro-6-[3-(4,5-dihydro-1-methylimidazol-2-yl)phenoxy]-4-[2,3-dihydroxypropoxy]-pyridin-2-yloxy)-4-hydroxybenzamidine (see international patent application WO 00/31068); ZK-807834 (see international patent application WO 97/29067); 1,4-diaza-4-(6-chloronaphthalene-2-ylsulfonyl)-6-(methoxymethyl)-7-oxa-1′-(pyridin-4-yl)spiro[bicyclo[4.3.0]-nonane-8,4′-piperidine]-2-one (see international patent application WO 01/02397); (S)-1-(4-aminoquinazolin-7-ylmethyl)-4-[2-(5-chlorothien-2-yl-oxy)acetyl]-3-methoxymethylpiperazin-2-one (see international patent application WO 00/32590); 3-(2-[4(2-aminosulfonylphenyl)benzoyl-amino]phenoxy)benzamidine (see international patent application WO 01/19788); and 4-(2-[4-(5-chloroindol-2-ylsulfonyl)-2-(pyrrolidin-1-ylcarbonylmethyl)piperazin-1-ylcarbonyl]thiazol-5-yl)pyridine N-oxide (see Japanese patent application No. JP 12143623); as well as the compounds of Example 7 of international patent application WO 98/21188, of Examples 3 and 6 of WO 99/57113, of Example 6 of international patent application WO 00/78747, of Examples 188, 211 and 167 of U.S. Pat. No. 6,080,767, of Examples 40, 54 and 55 of international patent application WO 99/33805, of Examples 5, 6, 8, 9, 10, 11, 12, 13, 15, 16 and 17 of international patent application WO 01/05784, of Examples 6, 7, 8, 9, 10, 11, 12, 13, 14, 15, 16, 17, 22, 23, 25, 26, 28, 29, 30, 31, 32, 33, 34, 38, 39, 40, 41, 42 and 43 of international patent application WO 01/12600, and of Examples 802 and 877 of international patent application WO 00/35886.

The term “condition where anticoagulant therapy is indicated” will be understood by those skilled in the art to include the following:

The treatment and/or prophylaxis of thrombosis and hypercoagulability in blood and/or tissues of animals including man. It is known that hypercoagulability may lead to thrombo-embolic diseases. Conditions associated with hypercoagulability and thrombo-embolic diseases which may be mentioned include inherited or acquired activated protein C resistance, such as the factor V-mutation (factor V Leiden), and inherited or acquired deficiencies in antithrombin III, protein C, protein S, heparin cofactor II. Other conditions known to be associated with hypercoagulability and thrombo-embolic disease include circulating antiphospholipid antibodies (Lupus anticoagulant), homocysteinemi, heparin induced thrombocytopenia and defects in fibrinolysis, as well as coagulation syndromes (e.g. disseminated intravascular coagulation (DIC)) and vascular injury in general (e.g. due to surgery).

The treatment of conditions where there is an undesirable excess of thrombin without signs of hypercoagulability, for example in neurodegenerative diseases such as Alzheimer's disease.

Particular disease states which may be mentioned include the therapeutic and/or prophylactic treatment of venous thrombosis (e.g. DVT) and pulmonary embolism, arterial thrombosis (e.g. in myocardial infarction, unstable angina, thrombosis-based stroke and peripheral arterial thrombosis), and systemic embolism usually from the atrium during atrial fibrillation or from the left ventricle after transmural myocardial infarction, or caused by congestive heart failure; prophylaxis of re-occlusion (ie thrombosis) after thrombolysis, percutaneous trans-luminal angioplasty (PTA) and coronary bypass operations; the prevention of re-thrombosis after microsurgery and vascular surgery in general.

Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis; the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease and the formation of atherosclerotic plaques, cerebral arterial disease, cerebral infarction, cerebral thrombosis, cerebral embolism, peripheral arterial disease, ischaemia, angina (including unstable angina), reperfusion damage, restenosis after percutaneous trans-luminal angioplasty (PTA) and coronary artery bypass surgery.

Preferred conditions include thrombosis.

In accordance with the invention, melagatran, Factor Xa inhibitors and derivatives of either, may be administered orally, intravenously, subcutaneously, buccally, rectally, dermally, nasally, tracheally, bronchially, topically, by any other parenteral route, or via inhalation, in the form of a pharmaceutical preparation comprising melagatran and/or Factor Xa inhibitor in a pharmaceutically-acceptable dosage form. Depending on the disorder, and the patient, to be treated, as well as the route of administration, the compositions may be administered at varying doses.

Preferred modes of delivery are systemic. For melagatran and derivatives thereof, preferred modes of administration are parenteral, more preferably intravenous, and especially subcutaneous. For prodrugs of melagatran, preferred modes of administration are oral.

In the therapeutic treatment of mammals, and especially humans, melagatran, Factor Xa inhibitors, and derivatives of either will generally be administered as a pharmaceutical formulation in admixture with a pharmaceutically-acceptable adjuvant, diluent or carrier, which may be selected with due regard to the intended route of administration and standard pharmaceutical practice.

Suitable formulations for use in administering melagatran and derivatives (including prodrugs) thereof are described in the literature, for example as described in inter alia international patent applications WO 94/29336, WO 96/14084, WO 96/16671, WO 97/23499, WO 97/39770, WO 97/45138, WO 98/16252, WO 99/27912, WO 99/27913, WO 00/12043 and WO 00/13671, the disclosures in which documents are hereby incorporated by reference.

Similarly, suitable formulations for use in administering Factor Xa inhibitors and derivatives (including prodrugs) thereof are described in the literature, for example as described in the prior art documents relating to Factor Xa inhibitors that are mentioned hereinbefore, the disclosures in which documents are hereby incorporated by reference. Otherwise, the preparation of suitable formulations, and in particular combined preparations including both melagatran/derivative and Factor Xa inhibitor/derivative may be achieved non-inventively by the skilled person using routine techniques.

The amounts of melagatran, Factor Xa inhibitor, or derivative of either, in the respective formulation(s) will depend on the severity of the condition, and on the patient, to be treated, as well as the compound(s) which is/are employed, but may be determined non-inventively by the skilled person.

Suitable doses of melagatran, Factor Xa inhibitors and derivatives of either, in the therapeutic and/or prophylactic treatment of mammalian, especially human, patients may be determined routinely by the medical practitioner or other skilled person, and include the respective doses discussed in the prior art documents relating to melagatran (or derivatives (including prodrugs) thereof), and to Factor Xa inhibitors, that are mentioned hereinbefore, the disclosures in which documents are hereby incorporated by reference.

In the case of melagatran, suitable doses of active compound, prodrugs and derivatives thereof, in the therapeutic and/or prophylactic treatment of mammalian, especially human, patients include those which give a mean plasma concentration of up to 5 μmol/L, for example in the range 0.001 to 5 μmol/L over the course of treatment of the relevant condition. Suitable doses may thus be in the range 0.1 mg once daily to 25 mg three times daily, and/or up to 100 mg infused parenterally over a 24 hour period, for melagatran, and in the range 0.1 mg once daily to 100 mg three times daily for prodrugs of melagatran including those specifically mentioned hereinbefore.

In the case of Factor Xa inhibitors, suitable doses for therapeutic or prophylactic purposes are in the range such that, for example, 10 to 500 mg is received, given if required in divided doses. In general lower doses will be administered when a parental route is employed, for example a dose for intravenous administration in the range, for example, 1 to 50 mg will generally be used. At continuous infusion, a dose of between 0.1 and 5 mg/kg/hour will generally be used.

In any event, the physician, or the skilled person, will be able to determine the actual dosage which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this invention.

When separate formulations are administered, the sequence in which the formulations comprising melagatran (or derivative thereof), and Factor Xa inhibitor (or derivative thereof), may be administered (i.e. whether, and at what point, sequential, separate and/or simultaneous administration takes place) may be determined by the physician or skilled person. For example, the sequence may depend upon many factors that will be evident to the skilled person, such as whether, at any time during the course or period of treatment, one or other of the formulations cannot be administered to the patient for practical reasons (e.g. the patient is unconscious and thus unable to take an oral formulation comprising either melagatran or Factor Xa inhibitor).

The method described herein may have the advantage that, in the treatment of conditions where anticoagulant therapy is indicated, it may be more convenient for the physician and/or patient than, be more efficacious than, be less toxic than, have a broader range of activity than, be more potent than, produce fewer side effects than, or that it may have other useful pharmacological properties over, similar methods known in the prior art for the treatment of such conditions.

The invention is illustrated, but in no way limited, by the following examples with reference to the figures in which:

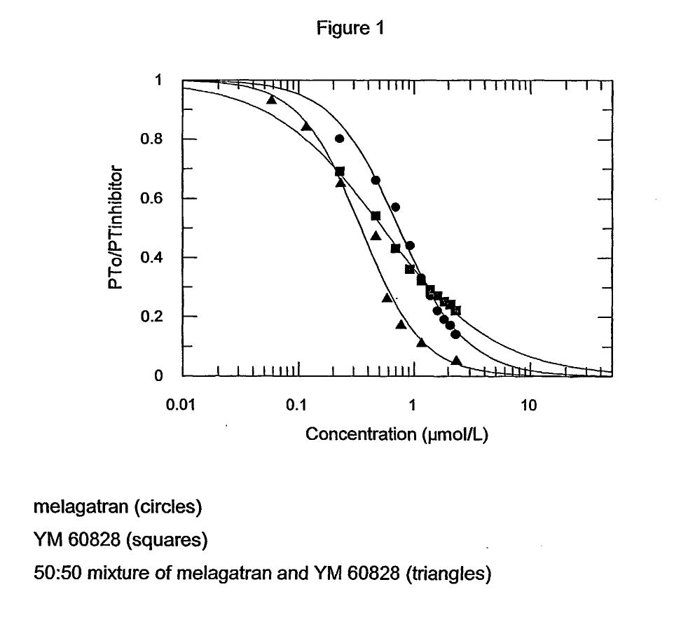

FIG. 1 illustrates dose-response curves for melagatran (circles), the Factor Xa inhibitor YM 60828 (squares), and a 50/50 mixture of melagatran and YM 60828 (triangles) in a prothrombin time clotting assay.

### EXAMPLE 1

4-{4-[4(5-Chloroindol-2-ylsulfonyl)piperazine-1-carbonyl]phenyl}-pyridine-1-oxide

To a stirred suspension of 1-(5-chloroindol-2-ylsulfonyl)4-[4-(4-pyridyl)benzoyl]piperazine (500 mg, 1.04 mmol) in dichloromethane (25 mL) was added 3-chloroperoxybenzoic acid (640 mg of 70-75% strength, 2.6 mmol, 2.5 mol eq.) and the reaction stirred for 2 hours at ambient temperature, at which time use of thin layer chromatography indicated complete reaction. The reaction mixture was washed sequentially with sodium metabisulphite solution (2×20 mL of 1M) and brine, and then dried (phase-separating paper). Evaporation gave crude product as a brown solid; crystallisation from ethanol gave 4-{4-[4-(5-chloroindol-2-ylsulfonyl)piperazine-1-carbonyl]phenyl}pyridine-1-oxide as a pale yellow crystalline solid.

¹H NMR (d₆DMSO): 3.0-3.3 (broad s, 4H), 3.4-3.9 (broad d, 4H), 7.0 ppm (s, 1H), 7.35 (d, 1H), 7.5 (m, 3H), 7.8 (m, 5H), 8.3 (d, 2H), 12.4 (s, 1H)

MS (M+H)+495/497

mp 265-267° C.

The preparation of 1-(5-chloroindol-2-ylsulfonyl)-4[(4-pyridyl)benzoyl]piperazine is described in Example 3 of international patent application WO 99/57113.

### EXAMPLE 2

Synergistic Effect of Melagatran and a Factor Xa Inhibitor in Prothrombin Time Clotting Assay

Human blood from healthy volunteers, both men and women, was collected in 50 mL conical plastic tubes containing 5 mL of 0.13 mol/L of buffered tri-sodium citrate. The blood was immediately chilled on ice and centrifuged at 2000×g for 20 minutes. Platelet poor plasma was then divided into fractions of 10 mL each and “spiked” with melagatran or with YM 60828 (a direct Factor Xa inhibitor; see Example 75 of international patent application WO 96/16940) to a final concentration of 2.33 μmol/L. The melagatran solution was then mixed with YM 60828 in serial proportions (9:1, 8:2, 7:3 etc.) as indicated in Table 1.

TABLE 1

| Melagatran | YM 60828 | Mixture |
|---|---|---|
| μmol/L | μmol/L | μmol/L |
| 2.33 | 0.00 | 2.33 |
| 2.10 | 0.23 | 2.33 |
| 1.86 | 0.47 | 2.33 |
| 1.63 | 0.70 | 2.33 |
| 1.40 | 0.93 | 2.33 |
| 1.17 | 1.17 | 2.33 |
| 0.93 | 1.40 | 2.33 |
| 0.70 | 1.63 | 2.33 |
| 0.47 | 1.86 | 2.33 |
| 0.23 | 2.10 | 2.33 |
| 0.00 | 2.33 | 2.33 |

100 μL of human plasma was pre-warmed to 37° C. for 180 seconds before coagulation was initiated by the addition of 200 μL pre-warmed assay reagent (Thromboplastin-S, ISI 1.1, Biopool Ume\dot{a}, Sweden). The prothrombin time (PT) was determined on a mechanical coagulometer, coupled to a CR-A calculator, CR 10 printer and a PR60 heat block (KC 10A Amelung, Germany). Each clotting test was performed in duplicate. The prothrombin time for melagatran and YM 60828 (2.33 μmol/L) was 95.2 and 64.9 seconds, respectively, whereas all mixtures of these inhibitors (still at a total concentration of 2.33 μmol/L) showed a surprisingly prolonged PT with a maximum around 300 to 310 seconds when the inhibitors were mixed at a proportion of 60-40% thrombin inhibitor and 40-60% factor Xa inhibitor. All results are shown in Table 2.

TABLE 2

| Melagatran | YM 60828 | Mixture | PT (sec) |
|---|---|---|---|
| μmol/L | μmol/L | μmol/L | seconds |
| 2.33 | 0.00 | 2.33 | 95.2 |
| 2.10 | 0.23 | 2.33 | 203.8 |
| 1.86 | 0.47 | 2.33 | 264.4 |
| 1.63 | 0.70 | 2.33 | 300.0 |
| 1.40 | 0.93 | 2.33 | 311.3 |
| 1.17 | 1.17 | 2.33 | 311.8 |
| 0.93 | 1.40 | 2.33 | 306.0 |
| 0.70 | 1.63 | 2.33 | 284.2 |
| 0.47 | 1.86 | 2.33 | 243.4 |
| 0.23 | 2.10 | 2.33 | 185.5 |
| 0.00 | 2.33 | 2.33 | 64.9 |

In order to test whether this is a “true” synergistic effect or not, full dose-response curves for each inhibitor as well as for a 50:50 mixture of melagatran and YM 60828 were constructed. Each inhibitor was tested at concentrations ranging from 0 to 2.33 μmol/L, as shown in Tables 3 (melagatran and YM 60828) and 4 (a 50:50 mixture of melagatran and YM 60828).

TABLE 3

| | |
|---|---|
| Plasma conc. | Prothrombin time (sec) |

| μmol/L | melagatran | YM 60828 |
|---|---|---|
| 0.00 | 14.4 | 14.9 |
| 0.23 | 18.1 | 21.6 |
| 0.47 | 21.8 | 27.7 |
| 0.70 | 25.4 | 34.4 |
| 0.93 | 32.6 | 41.8 |
| 1.17 | 43.2 | 46.8 |
| 1.40 | 53.8 | 52.1 |
| 1.63 | 65.3 | 55.5 |
| 1.86 | 76.3 | 60.2 |
| 2.10 | 87.2 | 62.1 |

TABLE 4
Plasma concentration (μmol/L)

| Total | Melagatran | YM 60828 | Prothrombin time |
|---|---|---|---|
| 0.00 | 0.00 | 0.00 | 14.2 |
| 0.058 | 0.029 | 0.029 | 15.2 |
| 0.117 | 0.058 | 0.058 | 17.0 |
| 0.233 | 0.117 | 0.117 | 21.7 |
| 0.466 | 0.233 | 0.233 | 30.2 |
| 0.583 | 0.291 | 0.291 | 53.8 |
| 0.777 | 0.388 | 0.388 | 85.4 |
| 1.165 | 0.583 | 0.583 | 134.7 |
| 2.330 | 1.165 | 1.165 | 300.0 |

In order to determine the relative potency of each inhibitor and a 50:50 mixture thereof, the concentration required to double the prothrombin time (IC₅₀) was determined as follows: The ratio of prothrombin time in control plasma (PT₀) to the prothrombin time in the presence of different inhibitor concentrations (PT_{inhibitor}) were plotted against the inhibitor concentration (log transformed) and fitted to sigmoidal dose-response curves from which IC₅₀ values were calculated according to the equation:

y=a/[1+(x/IC₅₀)^{s]}

where y=(PTO)/(PT) with a range from 1 to 0; a=maximal range i.e. 1, when PTₘₑₗₐ is equal to PT₀; s=the slope of the dose-response curve, x=log concentration of inhibitor, IC₅₀=the concentration of melagatran that doubles the prothrombin time. The calculations were processed on a PC using the software program GraFit® Erithacus Version 3.0 (Robin Leatherbarrow, Imperial Collage of Science, Technology & Medicine, London).

The dose-response curves for melagatran, YM 60828, and a 50:50 mixture of these inhibitors are shown in FIG. 1 from which IC₅₀ values were calculated to be 0.74, 0.54 and 0.34 μmol/L, respectively.

In order to test if the lower IC₅₀ value for the mixture, as compared to both melagatran and YM 60828 alone reflects a true synergistic effect, the results were applied to an equation for synergy, as defined by Beerenbaum:

dose A/A_{E}+dose B/B_{E}<1

in which A_{E} and B_{E} are equipotent concentrations of melagatran and YM 60828 (i.e. the IC₅₀ values for these compounds which were 0.74 and 0.54 μmol/L, respectively). Dose A and B are the concentration of melagatran and YM 60828 in the 50:50 mixture which gave an IC₅₀ value of 0.34 μmol/L, i.e. A and B is 0.17 μmol/L. Entering these constants into the above equation results in a value of 0.54, which demonstrates that the synergy criterion is fulfilled, and melagatran and YM 60828 exert a true synergistic effect in vitro when they are tested in a prothrombin time clotting assay.

1. A combination product comprising:
(A) melagatran or a pharmaceutically-acceptable derivative thereof; and
(B) a Factor Xa inhibitor or a pharmaceutically-acceptable derivative thereof,
wherein each of components (A) and (B) is formulated in admixture with a pharmaceutically-acceptable adjuvant, diluent or carrier.

2. A combination product as claimed in claim 1 which comprises a pharmaceutical formulation including melagatran or a pharmaceutically-acceptable derivative thereof, and a Factor Xa inhibitor or a pharmaceutically-acceptable derivative thereof, in admixture with a is pharmaceutically-acceptable adjuvant, diluent or carrier.

3. A combination product as claimed in claim 1 which comprises a kit of parts comprising components:
(a) a pharmaceutical formulation including melagatran or a pharmaceutically-acceptable derivative thereof, in admixture with a pharmaceutically-acceptable adjuvant, diluent or carrier; and
(b) a pharmaceutical formulation including a Factor Xa inhibitor or a pharmaceutically-acceptable derivative thereof, in admixture with a pharmaceutically-acceptable adjuvant, diluent or carrier,
which components (a) and (b) are each provided in a form that is suitable for administration in conjunction with the other.

4. A kit of parts as claimed in claim 3, wherein components (a) and (b) are suitable for sequential, separate and/or simultaneous use in the treatment of a condition where anticoagulant therapy is indicated.

5. A combination product as claimed in any one of claims 1 to 4, wherein the derivative of melagatran is a prodrug of melagatran.

6. A combination product as claimed in claim 5, wherein the prodrug is of the formula
R¹O₂C—CH₂—(R)Cgl-Aze-Pab-OH,
wherein R¹ represents linear or branched C₁₋₆ alkyl and the OH group replaces one of the amidino hydrogens in Pab.

7. A combination product as claimed in claim 6, wherein R¹ represents methyl, ethyl, n-propyl, i-propyl or t-butyl.

8. A combination product as claimed in any one of claims 1 to 7, wherein the derivative of the Factor Xa inhibitor is a prodrug of that inhibitor.

9. A method of making a kit of parts as defined in any one of claims 3 to 8, which method comprises bringing a component (a), as defined in any one of claims 3 to 8, into association with a component (b), as defined in any one of claims 3 to 8, thus rendering the two components suitable for administration in conjunction with each other.

10. A kit of parts comprising:
(I) one of components (a) and (b) as defined in any one of claims 3 to 8; together with
(II) instructions to use that component in conjunction with the other of the two components.

11. A method of treatment of a condition where anticoagulant therapy is indicated, which comprises administration of a combination product as defined in any one of claims 1 to 8 or a kit of parts as defined in claim 10 to a patient suffering from, or susceptible to, such a condition.

12. The use of a combination product as defined in any one of claims 1 to 8 or a kit of parts as defined in claim 10 for the manufacture of a medicament for the treatment or prophylaxis of a condition where anticoagulant therapy is indicated.

13. The use of melagatran or a pharmaceutically-acceptable derivative thereof and a Factor Xa inhibitor or a pharmaceutically-acceptable derivative thereof for the manufacture of a medicament for the treatment or prophylaxis of a condition where anticoagulant therapy is indicated.
